(19) United States
(12) Patent Application Publication
(10) Pub. No.: US 2011/0165068 A1
(43) Pub. Date: Jul. 7, 2011

(54) Liposome medicament, method of preparation and use thereof

(76) Inventors: Yanfang Liu, Xi' an (CN); Jia Liu, San Diego, CA (US)

(21) Appl. No.: 12/926,654

(22) Filed: Dec. 2, 2010

Related U.S. Application Data
(63) Continuation-in-part of application No. PCT/CN2008/071258, filed on Jun. 11, 2008.

(51) Int. Cl. A61K 51/00; A61K 9/127; A61K 39/44; A61K 38/43; A61K 31/7088; A61K 38/02; A61K 38/46; A61K 31/575; B82Y 5/00

(52) U.S. Cl. 424/1.21; 424/450; 424/178.1; 424/94.1; 514/44 R; 514/1.1; 424/94.6; 514/182; 514/171; 977/798; 977/907

(57) ABSTRACT

This invention relates to a liposome medicament, and, more particularly, a liposome medicament with targeting function. The liposome medicament includes a medicament that is encapsulated in a liposome, and the medicament contains an effector molecule or an effector molecule that is combined with a first ligand, and a second ligand that is connected onto the surface of the liposome. The first ligand and/or the second ligand can specifically bind to target tissues or target cells to be treated. Preferably, the first ligand and/or the second ligands are antibodies, such as monoclonal antibodies. This invention also relates to a method of preparation of the liposome medicament and use of the medicament for treatment of diseases especially tumors. This invention further relates to a pharmaceutical composition of the liposome medicament and a pharmaceutically acceptable carrier.

### CROSS-REFERENCE TO RELATED APPLICATIONS

This is a Continuation-in-Part Application of and claims the benefit of priority under 35 U.S.C. 120 to PCT Application No. PCT/CN2008/071258, filed Jun. 11, 1998, published as WO 2009/149599 on Dec. 17, 2009, the disclosure of which is incorporated by reference herein in its entirety.

### FIELD OF THE INVENTION

The present invention relates to liposome medicaments, and, more particularly, to a liposome medicament with targeting function, wherein the liposome encapsulates a targeting medicament having an effector or an effector conjugated with a first ligand, to the surface of the liposome is bound a second ligand, and wherein the first ligand and the second ligand can be the same or different. The invention also relates to a method of manufacture of liposome medicaments and a method for treatment of diseases especially tumors. The invention further provides a pharmaceutical composition containing a liposome medicament and a pharmaceutically acceptable carrier.

### BACKGROUND OF THE INVENTION

As the tumor treatments by conventional surgery, chemotherapy and radiation showed very limited effects, there have been tremendous efforts in many countries to develop new drugs and look for new approaches for tumor treatments. Recent development in biotechnologies has enabled to obtain certain breakthrough results in researches of therapeutic antibodies, especially those for tumor treatment. One of the approaches is to link antibodies to chemotherapeutic agents, which is known as antibody drug conjugation (ADC). ADCs are considered attractive, safer alternatives to conventional chemotherapy because they combine the specificity of antibodies with the potency of cytotoxic molecules, delivering them directly to their intended targets and, theoretically, avoiding systemic toxicity. This system, however, has its limitation in terms of high drug loading capacity. New approaches using pharmaceutical carriers as polymers, liposomes, and micelles have provided clear advantages, such as high loading capacity, possible size control, permeability of drug carrier systems and use relatively small number of vector molecules to deliver substantial quantities of drugs to the target. Monoclonal antibodies can be applied to liposomes that encapsulated pharmaceutical agents or toxins, i.e., immune liposome toxins. Efforts have also been made to use genetically engineered antibodies, especially single-chain antibodies to cross-link onto the surface of liposomes that encapsulate therein respectively pharmaceutical agents, toxins, cytokines, genes and antisense nucleic acids, to form also immune liposome toxins.

### SUMMARY OF THE INVENTION

In the present invention, it was unexpectedly found that the novel targeting liposomes that have very high specificity to target tissues can be prepared by using liposome technology and combining effectors, such as therapeutically active agents, with ligands that are capable of targeting the target tissues.

The present invention, in one aspect, provides a targeting liposome medicament, wherein a liposome encapsulates a medicament including an effector, and outside of the liposome is connected with a ligand. The ligand can specifically bind to target tissues or target cells of the subjects to be treated or handled.

The present invention, in another aspect, provides a double-targeting liposome medicament, wherein a liposome encapsulates a targeting medicament including a first ligand combined with an effector, and outside of the liposome is connected with a second ligand. The first ligand and the second ligand can specifically bind to target tissues or target cells of the subjects to be treated or handled.

The first ligand and the second ligand can be the same or different. Preferably, the first ligand and/or the second ligand are antibodies, particularly monoclonal antibodies, or their antigen binding fragments, such as Fab fragments, F(ab′)₂ fragments, single-chain antibodies, and humanized antibodies.

In the present invention, the first ligand and the second ligand can be connected to effector molecules and the surface of liposomes through varieties of possible ways. In one embodiment, the first ligand is bound covalently or non-covalently with an effector molecule to form a fusion protein. In another embodiment, the second ligand is conjugated or coupled to the surface of liposome.

The present invention, in another aspect, provides a method of preparation of liposome medicaments with desirable targeting function.

The present invention also relates to pharmaceutical compositions of liposome medicaments with desirable targeting function, and their uses in the treatment of diseases, especially tumors.

The present invention also provides a method for treatment of diseases, including applications of liposome medicaments or pharmaceutical compositions of the invention to patients who need treatment.

The present invention also provides a method for diagnosis of diseases, including applications of liposome medicaments or pharmaceutical compositions of the invention to subjects.

### BRIEF DESCRIPTION OF THE DRAWINGS

FIG. 1 illustrates the amino acid sequence and its coding sequence (SEQ ID NO: 1&2) of genetically engineered anti-hepatoma single-chain antibody scFv25;

FIG. 2 illustrates the coding sequence of single-chain antibody HdcFv25 obtained after scFv25 was humanized and disulfide stabilized; and

FIGS. 3A and 3B illustrate the amino acid sequence and its corresponding coding sequence of humanized and disulfide stabilized single-chain antibody hdcFv25.

### DETAILED DESCRIPTION OF THE INVENTION

As used herein, the term “ligand” refers to any molecular entity which can be specifically combined with target cells, tissues and/or organs, especially tumor-tissues, such that this term should be broadly construed. Although ligands can be combined with target cells through cell receptors on the surface of the target cells, but no such limitation is to be inferred in this invention. In one embodiment, for example, the ligand can be combined with target cells or target tissues, such as tumor cells or tissues, through related antigens of the tumors.

The first ligand and the second ligand, in accordance with embodiments of the present invention, can be the same or different, and can be selected independently from any desirable types. An ordinarily skilled technician should know how to select appropriate first ligand and/or second ligand according to specific target tissues to be treated. Preferably, the first ligand and/or the second ligand are antibodies, particularly monoclonal antibodies, or their antigen-binding fragments, such as Fab fragments, F(ab′)₂ fragments, single chain antibodies and humanized antibodies. In one embodiment, for example, the first ligand and/or the second ligand are independently monoclonal antibodies or antigen-binding fragments selected from the group including: CD19, CD20, CD22, CD33, epidermal growth factor receptor (EGFR), MUC-1, prostate specificity antigen (PSA), prostate specificity membrane antigen (PSMA), oncogene c-erbB2 product, ganglioside GD3, and GM2. An ordinarily skilled technician should be able to understand that this invention is not limited by the specific examples illustrated herein.

In a preferred embodiment, the ligands are antibodies or antibody fragments. Usable antibody fragments include, for example, fragments of Fab, Fab′, F(ab′)₂, scFv, and dsFv. The antibodies or antibody fragments can be from any species, including mouse, rat, rabbit, hamster, and human. The present invention covers also the use of chimeric antibody derivatives, i.e. antibody molecules with non-human animal variable regions and human constant regions being combined. Chimeric antibody molecules may include humanized antibodies, which contains antigen binding regions of the antigens of such as mouse, rat or other species, and human constant regions, and which can be prepared by using certain conventional methods in the art. For example, humanized antibodies can be prepared by referring to the document of EP-B 10 239400. In addition, humanized antibodies may be also obtained commercially. It is expected that the immunogenicity of chimeric antibody in human subjects is lower than that of corresponding non-chimeric antibody. In order to improve their properties, humanized antibodies can be further stabilized, for example, through the procedures described in the document of WO 00/61635.

Specificity antibodies or antibody fragments can be also prepared by screening encoded immunoglobulin genes or their partial expression library, wherein the expression library expresses in bacteria the peptides produced through encode of the nucleic acid molecules of the protein. For example, an expression library of bacteriophage can be used to express in bacteria the entire Fab fragment, VH region, and FV region. Alternatively, SCID-hu mice can also be used, with the mouse model developed by Genpharm for example, to produce antibodies or their fragments.

The first ligand and/or the second ligand of the targeting liposome of the present invention can be partially independently derived from immunoglobulin. For example, a ligand can be obtained by using standard techniques known in the art to modify immunoglobulin scaffold. In another non-limiting embodiment, structural regions of immunoglobulin (such as heavy-chain and/or light-chain variable regions) can be connected with non-immunoglobulin scaffolds. In addition, a ligand can be prepared through chemical reactions or genetic design. Therefore, in non-restrictive examples, targeting medicaments may include (1) polypeptides derived from immunoglobulin (antibodies selected from antibody libraries for example) or their variants, or their specific binding target tissues and/or cells such as tumor cells, and (2) effector molecules, such as toxins, or their variants. The polypeptide ligands of immunoglobulin can be redesigned to change their binding characteristics towards targets such as tumor related molecules, or, for example to improve their physical characteristics.

The ligands of targeting liposomes of the present invention can also be partially independently not based on immunoglobulin. In some embodiments, for example, the first ligand and/or the second ligand can be non-immunoglobulin polypeptides (e.g., Affibody(R)) of specific binding target tissues such as tumor cells or their variants. Accordingly, the described targeting medicaments may include: (1) Non-immunoglobulin polypeptides (e.g., Affibody(R)) of specific binding target tissues, such as tumor cells or their variants, and (2) effector molecules, such as toxins or their variants. Such non-immunoglobulin ligands can be designed as molecules related to binding target tissues such as target tumors. In addition, non-immunoglobulin polypeptide ligands can be prepared so as to have desirable affinity and be able to withstand various physical conditions including extreme pH ranges and relatively high temperatures.

It is highly desirable that, as used in pharmaceutical compositions, the non-immunoglobulin polypeptides of this invention are designed to have a relatively long half-life under certain physiological conditions (such as at 37° C. and with the existence of peptidase). In addition, such molecules or their variants can be small in size and possess good solubility and appropriate foldability, and can proceed expression in readily utilizable and less expensive bacterial systems for commercial manufacturing in reasonable scales. The design of non-immunoglobulin polypeptides is comprehensible within the capacity of an ordinarily skilled technician. The techniques for design, production and selection of binding ligands can be generally referred to, for example U.S. Pat. Nos. 5,831,012 and 6,534,628, where certain adoptive modifications could be included.

In another embodiment, the first ligand and/or the second ligand of the invention can independently be epitope binding peptides. Examples of the epitope binding peptides include, but not limited to, ligands containing fibronectin type III domain. Also, the affinity library based on protein A can be used to identify epitope binding peptides, while in the present invention such library can be used to pick out the peptides that bind selectively with the target tumor cells.

The first ligand and/or the second ligand of the present invention can also independently be other types of binding molecules. Such known binding molecules include, but not limited to, binding molecules based on assembly of repeat protein domain. In accordance with the present invention, the library of randomly assembled repeat domain can be used to pick out the ligands that bind selectively with target tissues such as tumor cells.

Preferably, the first ligand and/or second ligand can specifically bind to tumor tissues through tumor markers, the tumor markers including but not limited to, for example, carcinoembryonic antigen, prostate specific antigen, urinary tumor associated antigen, fetal antigens, tyrosinase (p97), gp68, TAG-72, HMFG, sialic acid Lewis antigen, MucA, MucB, PLAP, estrogen receptor, laminin receptor, erb B, and pi 55.

More preferably, the first ligand and/or the second ligand are monoclonal antibodies against liver cancer. The monoclonal antibodies can be prepared by the methods known to an ordinarily skilled technician in the art, such as hybridoma technology. In one embodiment, the first ligand and/or the second ligand can be independently monoclonal antibodies, which are derived from HAb25 hybridomaor derivatives of the monoclonal antibodies. These derivatives respectively have heavy-chains and light-chains that are different from the corresponding sequences of HAb25 monoclonal antibody due to respectively substitution, insertion, deletion and/or addition of 1-20, preferably 1-15, more preferably 1-10, even more preferably 1-8, particularly 1-5, for example 1, 2, 3 or 4 of amino acid residues, but retain the antigen/target tissue binding affinity of HAb25 monoclonal hybridoma antibodies. Further preferably, the first ligand and/or the second ligand are single-chain antibody scFv25 which is derived from a HAb25 hybridoma monoclonal antibody and have an amino acid sequence shown in SEQ ID NO: 1. The coding sequence of the single-chain antibody ScFv25 is shown in SEQ ID NO: 2. Alternatively, the first ligand and/or the second ligand can be a variant of the single-chain antibody scFv25, which has an amino acid sequence that is different from SEQ ID NO: 1 due to substitution, insertion, deletion and/or addition of 1-20, preferably 1-15, more preferably 1-10, even more preferably 1-8, particularly 1-5, for example 1, 2, 3 or 4 amino acid residues, but retains the antigen/target tissue binding affinity of the single-chain antibody.

It is known that single-chain antibodies can be humanized and disulfide stabilized to improve their properties for clinical applications. In a preferred embodiment, therefore, the first ligand and/or the second ligand can be independently a single-chain antibody that has been humanized and/or disulfide stabilized, such as a humanized monoclonal antibody hscFv25 with an amino acid sequence shown in SEQ ID NO: 3 or a humanized and disulfide stabilized single-chain antibody hdcFv25 with an amino acid sequence shown in SEQ ID NO: 5. Alternatively, the first ligand and/or the second ligand can be independently an amino acid sequence that is different from SEQ ID NO: 3 or 5 due to substitution, insertion, deficience and/or addition of 1-20, preferably 1-15, more preferably 1-10, even more preferably 1-8, particularly 1-5, for example 1, 2, 3 or 4 amino acid residues, but retains the antigen/target tissue binding affinity of the single-chain antibody hscFv25 or hdcFv25.

As described herein, the term “medicaments,” as used interchangeably with the term “effectors” or “biologically or therapeutically active agents,” refers to any pharmaceutical agents which are capable of having in vivo or in vitro physiological effects (such as those for treatment or prevention) to biological systems such as prokaryotic or eukaryotic cells. Examples of the medicaments include, but not limited to, chemotherapeutic agents, toxins, radiotherapy agents, radiation sensitizing agents, gene therapy carriers, antisense nucleic acid constructs, transcription factor decoies, imaging agents, diagnostic agents, pharmaceuticals known to interact with cellular proteins, vaccines, peptides and polynucleotides such as inhibition RNA, antisense RNA, siRNA, and genes.

In one embodiment, the “effectors” are any molecules which are capable of having direct or indirect desired functions in the target cells, such as therapy active agents. In one embodiment, the effectors are therapeutically active agents, which can be any active agents suitable to treat or prevent therapeutic targets and diseases, for example, a medicament such as doxorubicin, cytotoxic agent such as PE38, cytokines such as TNF, or radioisotope such as ¹³¹I, therapeutic protein.

In a non-restrictive embodiment, the therapeutically active agents are cytotoxic agents, including such as bacterial and plant toxins. Examples of cytotoxic agents include, but not limited to, pokeweed antiviral proteins, saponins, gelonin, ricins, abrins, pseudomonas exotoxin A, diphtheria toxin a-sarcin, bouganin, bryodins, restrictocin, Shiga toxin, and their variants.

In another non-restrictive embodiment, the therapeutically active agents are DNA damaging toxins. The toxins include, but not limited to, enediynes (such as calicheamicin and esperamicin) and non-enediyne small molecule agents such as bleomycin, methidiumpropyl-EDTA-Fe (II)). Other toxins that can be used in the present invention include daunorubicin, doxorubicin, stallimycin A, cisplatin, mitomycin C, ecteinascidin, bleomycin, and peplomycin.

In yet another non-limiting embodiment, the therapeutically active agents are tubulin damaging toxins. The toxins include, but not limited to, rhizoxin/maytansine, paclitaxel, vincristine, vinblastine, and colchicine.

In the present invention, the active agents can be also other kinds known to an ordinarily skilled technician in the art. The information about other therapeutically active agents can be referred in various literatures in the field.

In another embodiment, the effectors are RNases, which are enzymes capable of decomposing RNA, and are non-toxic to normal cells but toxic to tumor cells.

In a further embodiment, the effectors are diagnostic agents, such as markers. The markers include, such as fluorescent markers, enzyme markers, radioactive markers, nuclear magnetic resonance active markers, light-emitting markers, or chromophore markers. The diagnostic agents, after being targeted to the target tissue by a first ligand, can achieve diagnosis purposes by ways of, for example, imaging method.

As described above, the targeting medicaments of the present invention include an effector capable of having desired functions, such as cytotoxicity, to the cells. Further, for enhancing the targeting ability, the targeting medicaments of the present invention can also include: (1) A first ligand specifically bond with target cells; and (2) an effector capable of having desired functions, such as cytotoxicity, to the cells. The effectors, such as therapeutically active agents, can be bond with the first ligand through any of various suitable means, such as that of conjugation or non-conjugation. For example, the first ligand can bind with the effector by chemical or recombinant methods. The chemical methods of preparing fusions or conjugates, as well known in the field, can be employed to prepare the targeting agents of the present invention. The methods of binding the first ligand and the effector must allow the first ligand to connect with the effector without interfering with the ligant's ability to combine with target molecules of the target tissues such as tumor cells.

According to one embodiment of the present invention, there can be one or multiple of effectors, all or partially, to couple/bind with the first ligand through the mechanisms of, for instance, covalent binding, affinity binding, embedding, coordinative binding, chelating, or complexing. Wherein, covalent binding can be achieved by direct condensation of existing side chains or by incorporation of external bridging molecules. Many bivalent or multivalent agents, which include, but not limited to, carbodiimides, diisocyanates, and glutaraldehydes, can be used to make protein molecules coupling with, for example, other proteins, peptides, or amino functional groups.

In some embodiments, the antibodies are firstly derivatized, and effectors are then attached to the resulting derivatives. Appropriate cross-linking agents in this aspect include, for example, SPDP (N-succinimidyl-3-(2-pyridyl-disulfenyl)propionate) and SMPT (4-succinimidyl-oxocarbonyl-methyl-(2-pyridine-dimercapto)toluene).

In other embodiments, the first ligand and the effectors are both proteins, and they can be bound together by using the techniques known in the art. There are hundreds of cross-linking agents that are known and can be used to bind the two proteins together. The cross-linking agents are usually selected on the basis of the first antibody or ligand and the active functional groups inserted or utilizable on the effectors. If there are no any active functional groups, then photo-activatable cross linkers can be used. In some cases, it may be desirable to have spacer groups between the first ligands and the effectors. Some known cross-linking agents include homobifunctional group agents: glutaraldehyde, adipic acid dimethyl ester and bi(aza-benzidine), and heterobifunctional group agents: m-maleimidebenzoyl-N-hydroxysuccinimide and sulfo m-maleimidebenzoyl-N-hydroxysuccinimide.

The fusion of the first ligand proteins and the effector proteins can also be prepared through recombinant DNA technology. In this case, the DNA sequences that code the first ligand proteins and the DNA sequences that code the effectors can be fused together to obtain chimeric DNA molecules. Then the chimeric DNA sequence is transfected into host cells that express fusion proteins of the first ligands and effectors, and the fusion proteins are recovered and purified from the culture by using the techniques known in this field.

In another embodiment, the effectors are radionuclides, which generally can be coupled through chelation to the first ligands of the present invention. For example, in the case of metallic radionuclides, bifunctional chelating agents are often used to connect the isotopes and the first ligands. Usually, the chelating agent attaches firstly to the first ligand, and then the complex of the chelating agent and the first ligand contacts with metal radionuclide. There are many known bifunctional chelating agents suitable for this purpose, which include, for example amino acids of the series of diethylene triamine pentaacetic acid (DTPA), as described in U.S. Pat. Nos. 5,124,471, 5,286,850 and 5,434,287, the disclosures of which are incorporated herein by reference.

In the present invention, the targeting medicaments are encapsulated in the liposomes to facilitate the delivery of the medicaments. Any suitable liposomes could be employed in the present invention. It should be understood that liposome is referred to the structure of lipid membrane with water encapsulated therein. Except as otherwise noted, such structure can have one or more layers of lipid membranes, although liposomes usually contain only one layer of membrane. Mono layered liposomes are herein referred to as of “single layered,” and the multiple layered liposomes are referred to as of “multi-layered.” In the present invention, liposomes can be in any suitable sizes, for example, 1 nm-50 μm, preferably 10 nm-10 μm, more preferably 30 nm-1 μm, especially 50-500 nm. In one embodiment, the liposomes are nano-liposomes.

Liposomes used in the present invention are preferably formed from lipids, which can form relatively stable vesicles while combining. There are many types of lipids known in this field that can be used to form this type of liposomes. The preferred species include, but not limited to, neutral and negatively charged phospholipids or nerve sheath lipids and sterols, such as cholesterol. For the selection of lipids, the stability in the bloodstream and the size of liposomes should be considered.

In the present invention, the liposomes include preferably sphingomyelins and cholesterols, wherein the ratio of sphingomyelins and cholesterols can be varied, usually from 75:25 to 30:50 mol/mol, preferably from about 70:30 to about 40:45 mol/mol. Otherwise, the ratio of phospholipid (SPC) and cholesterol (Chol) is, as measured by weight, preferably 10:1 to 1:1, more preferably 8:1 to 2:1, even more preferably 6:1 to 3:1, for example 5:1 to 4:1. When necessary, the liposomes of the present invention can also contain other types of lipids, such as, to prevent the lipids from being oxidized or prevent the liposomes from being attached with ligands on the surface thereof, cholesterol polyethylene glycol ester, the ratio of which incorporated as measured by molar number of phospholipid can be 1-15%, preferably 3-10%, more preferably 5-8%, such as 6%. Detailed descriptions of this type of liposomes can be found in U.S. Pat. No. 5,814,335, the disclosure of which is incorporated herein by reference in its entirety.

Varieties of methods can be used to prepare liposomes, for example, as described in U.S. Pat. Nos. 4,235,871 and 4,501,728, the contents of which are incorporated herein by reference in their entirety. The process of preparation of liposomes usually includes the following steps: Mixing the lipid components in an organic solvent, drying and reconstructing the liposomes in an aqueous solvent, and determining the size of the liposomes.

The targeting agents can be introduced into liposomes in either passive or active fashion. The passive introduction usually requires adding medicaments in the buffer in the process of reconstruction. This makes medicaments to be trapped inside the liposomes, and remain there if medicaments are insoluble in lipids and vesicles kept intact. Descriptions of such methods can be found, for example, in WO 95/08986, which is incorporated herein by reference in its entirety.

The active introduction can be proceeded in a variety of ways, while the use of trans-membrane pH or ion gradients can make encapsulation ratio to reach 100%. Such methods, as should be known to an ordinary skilled technician, all include establishment of gradients of some type. The gradients may facilitate the delivery of lipophilic components into the liposomes.

After the formation of liposomes, a second ligand can be bond or coupled onto the surface of the liposomes. Such methods are known to the ordinary skilled technicians in the art. The second ligand can be connected directly or through a linker with the liposomes. The linkers used in the present invention are also known to the ordinary skilled technicians, and include for example, carbodiimide and glutaraldehyde.

The targeting liposome medicaments of the present invention can be administered alone or in combination with other pharmaceutical agents or biologically active agents. Examples of the other pharmaceutical agents or biologically active agents include, but not limited to, antioxidants, free radical scavengers, peptides, growth factors, antibiotics, bacterial inhibitors, immuno-suppressants, anticoagulants, buffers, anti-inflammatory agents, antipyretics, analgesics, steroids, and corticosteroids. The treatment may also include surgery and/or chemotherapy. For example, the targeting liposomes can be administered in combination with radiotherapy and cisplatin (Platinol), fluorouracil (5-FU, Adrucil), carboplatin (Paraplatin) and/or paclitaxel (Taxol).

In one embodiment, the targeting liposome medicaments of the invention are used along with conventional radiotherapy. In such combined application, radiation treatment can be performed in lower doses or lower frequencies to decrease, for example, the radiation related side effects.

In another embodiment, the targeting liposome medicaments of the present invention can be administered in combination with one or more types of cytokines, while the cytokines include, but not limited to, lymphokines, tumor necrosis factor, tumor necrosis factor-like cytokine, lymphotoxin, interferon, macrophage inflammatory protein, monocyte-granulocyte colony stimulating factor, interleukin (including, but not limited to, interleukin-1, interleukin-2, interleukin-6, interleukin-12, interleukin-15 and interleukin-18), and their variants, and further pharmaceutically acceptable salts thereof.

In yet another embodiment, the targeting liposome medicaments of the present invention can be used in combination with cancer vaccines, while the cancer vaccines include, but not limited to, autologous cells or tissues, non-autologous cells or tissues, carcinoembryonic antigens, alpha fetoproteins, human chorionic gonadotropins, BCG live vaccines, melanocyte lineage proteins, and mutant tumor specific antigens.

In still yet another embodiment, the targeting liposome medicaments of the present invention can be used along with hormone therapy, while the hormone therapy include, but not limited to, the applications of hormone agonists, hormone antagonists (e.g., flutamide), tamoxifens, leuprolide acetates (LUPRON), and steroids (e.g., dexamethasone, retinoid, betamethasone, cortisol, cortisone, prednisone, dehydrotestosterone, glucocorticoid, mineralocorticoid, estrogen, testosterone, and progesterone).

In a further embodiment, the targeting liposome medicaments of the present invention can be used along with gene therapy treatment to treat or prevent diseases such as cancer.

The targeting liposome medicaments of the present invention can be contained in pharmaceutical compositions or drugs. The pharmaceutical compositions suitable for direct administration include freeze-dried powders, and aqueous or non-aqueous sterile injectable solutions or suspensions, and may further contain antioxidants, buffers, antimicrobial agents, and the solutes that are capable of causing the pharmaceutical compositions and the blood of anticipated recipient to be substantially isotonic. Such compositions may also contain other components, for example, water, alcohols, polyols, glycerols, and vegetable oils. The injection solution or suspension can be prepared from such as sterile powders, granules, and tablets. The targeting liposome medicaments of the present invention can be provided in the form of, for example, freeze-dried powder. The freeze-dried powders can be redissolved in sterile water or saline solution before being administered to patients.

The pharmaceutical compositions of the present invention may include pharmaceutically acceptable carriers. The suitable pharmaceutically acceptable carriers include chemically essentially inert non-toxic composition, which does not interfere with the effectiveness of biological activity of the pharmaceutical compositions. Suitable pharmaceutical carriers include, but not limited to, water, salt solution, glycerin, ethanol, N-(1-(2,3-dioleoyloxy)propyl)-N,N,N-trimethyl ammonium chloride (DOTMA), dioleoyl phosphatidyl ethanol amine (DOPE), and liposomes. Such compositions should contain a therapeutically effective amount of targeting liposome medicaments and an appropriate amount of carriers to provide a form suitable for direct administration to patients.

In another embodiment, the pharmaceutical compositions include targeting liposome medicaments of the present invention and one or more additional therapeutic agents such as cancer therapeutic agents, optionally in a pharmaceutically acceptable carrier.

The pharmaceutical compositions of this invention can be administered in a variety of appropriate ways, including, but not limited to, intra-arterial, intramuscular, intravenous, nasal, and oral routes. In a specific embodiment, the pharmaceutical compositions of the invention can be administered locally to an area in need of treatment. The local administration can be achieved by, for example, local infusion during a surgical procedure, injection or catheter.

In some embodiments of the invention, the pharmaceutical compositions are administered directly to the target tissues or target cell areas such as tumor cell area, for example, by local infusion during surgery, local application (for example, used along with wound dressing after surgery), injection, catheter, suppository, or implant. The implants can be of porous, non-porous or gelatin-like materials, including membranes or fibers. The suppositories generally contain 0.5-10 wt % of active ingredients.

In other embodiments, a controlled release system can be placed in the vicinity of the target tissues or target cells. For example, a micro-pump can be used to deliver controlled doses directly into the vicinity of the target tissues or target cells, and thus precisely adjust the timed release and concentration of the pharmaceutical compositions.

The invention also provides a kit, which contains an effective amount of the targeting liposome medicaments of the present invention, optionally one or more companying active agents such as therapeutic active agent or diagnostic agent, and instructions for use as well.

In the present invention, target cells, tissues or organs to be treated or diagnosed include, but not limited to, prostate tissue, ovarian tissue, colon tissue, epithelial tissue, blood cells, lung tissue, liver tissue and pancreas tissue, or tumor in the corresponding cells. Ordinary skilled technicians can choose suitable first ligands and second ligands based on type of the tissues, diseases and conditions to be treated and diagnosed. In one embodiment, the diseases or conditions include diseases of excessive proliferation, tumors for example which include such as bladder cancer, colon cancer, liver cancer, lung cancer, stomach cancer, prostate cancer, breast cancer, brain tumor and skin cancer; and arteriosclerotic psychosis.

According to one aspect of the present invention, the targeting liposome medicaments and/or other therapeutically active agents are delivered to patients by direct application. Therefore, the targeting liposome medicaments of the present invention and/or other therapeutically active agents can be administered, for example, by one or more direct injections into the target tissues such as tumors, intratumoral perfusion continuously or intermittently into the target tissues, introduction into a reservoir of the targeting liposome medicaments, incorporation of a release device into the targeting tissues such as tumor, and/or direct application to the target tissues such as tumors. In the present invention, the application of drugs into “the inside of tumors” also includes introducing the targeting liposome medicaments and/or other cancer therapeutic agents into the tumor region or into the blood vessel or lymphatic vessel that is basically directly connected to tumor region. In each case, the pharmaceutical composition is in an amount that is sufficient to achieve the intended treatment purpose, and, when necessary, also includes a pharmaceutically acceptable carrier.

It can be expected that the targeting liposome medicaments of the present invention can be introduced into the inside of tumors, while any other cancer therapeutic agents on the other hand can be delivered to the patients through other routes, intravenously for example. In addition, for the purpose of delivering multiple cancer therapeutic agents to the patients, the targeting liposome medicaments of the present invention and one or more of the cancer therapeutic agents can be delivered into the inside of the tumors, while other cancer therapeutic agents can be delivered via other routes, intravenously or orally for example.

The invention also provides a method for treatment of diseases, which includes administering the liposome medicaments or pharmaceutical composition of this invention to patients who need treatment. In one embodiment, the method also includes applying, sequentially or simultaneously, to patients with other therapies such as hormone therapy and radiation therapy.

The invention further provides a method for diagnosis of diseases, which includes administering the liposome medicaments or pharmaceutical composition of this invention to subjects. Preferably, the method also includes the step of forming image of subjects.

### EXAMPLES

The present invention could be better understood with reference to the following exemplary embodiments. The embodiments herein are for illustration only and not intended to limit the scope of the present invention.

### Example 1

### Preparation of the Nucleotide Sequence that Encoding Single-Chain Antibody scFv25

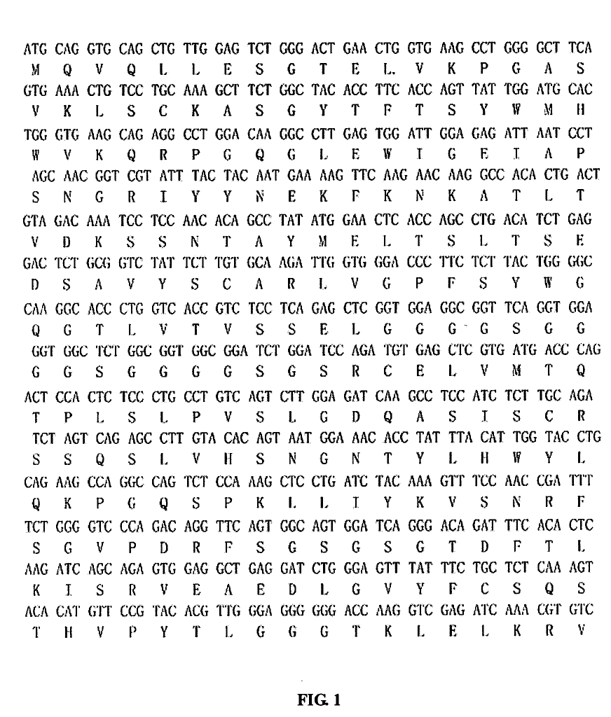

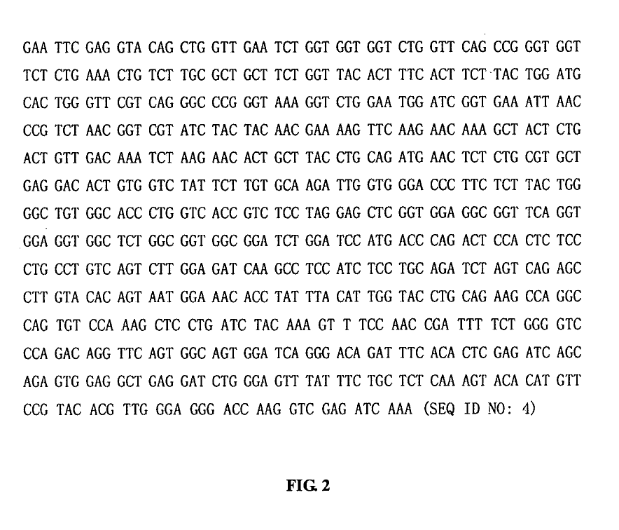

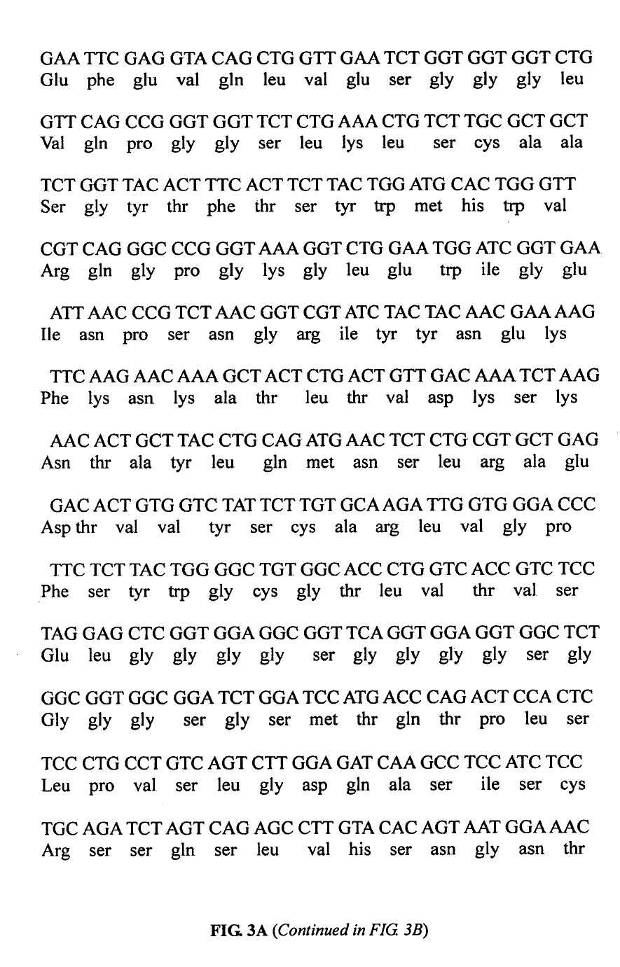

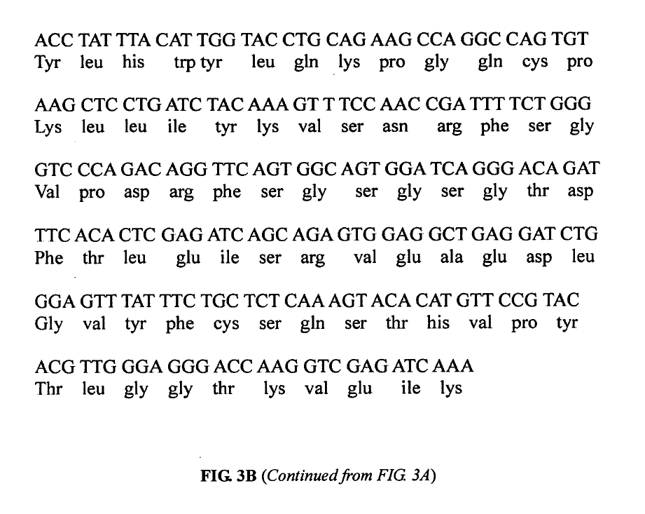

In reference to the sequence shown in SEQ ID NO: 2, the nucleotide sequence of encoding single-chain antibody scFv25 is prepared through chemical synthesis, and is cloned into pUC19 plasmid (Novagen Company) to form and express as pUC19 mscFv25. Experimental results showed that the use of mouse single-chain antibody mscFv25 (also referred herein as single-chain antibody scFv25) brought an increase by 84% in the image developing capability of human liver tumor that was transplanted into nude mice (tumor/liver ratio is 9.6). The amino acid sequence of single-chain antibody scFv25 is shown as SEQ ID NO: 1 and its sequence of coding nucleotide is shown as SEQ ID NO: 2, as illustrated also in FIG. 1. The single-chain antibody scFv25 is humanized to obtain expression vector pUC19hscFv25. Wherein, the humanized single-chain antibody scFv25 is called hscFv25. Furthermore, with pUC19hscFv25 as template and by using primer of

5′CCGCTCGACCTGGAGACGGTGACCAGGATGCCCAGCCCCA 3′, the position 105 of amino acids of V_{H} is changed into cys, and the position 43 of amino acids of V_{L} is changed also into cys, so as to obtain the humanized and disulfide stabilized single-chain antibody coding sequence. The humanized and disulfide stabilized single-chain antibody is called hdcFv25. The resultant encoding sequence is cloned into the expression vector pET15 bs between the sites of NcoI and NotI, to obtain expression vector pET15b-hdcFv25, which is transformed into BL21(DE3) so as to express hdcFv25 protein.

With the above expression vector pET15b-hdcFv25 as template, and hdcFv25 BACK: 5′ATAGTTTAGCGGCCGCTTTGATCTCGACCTGGTCCC3′ (SEQ ID NO: 7) and FOR: 5′CGGAATTCATGACCCAGACTCCACTC 3′ (SEQ ID NO: 8) as primers, hdcFv25 is PCR amplified, double enzyme digested by EcoR I and Not I, and thus DNA is recovered. Then, pTIH is double digested by EcoRI and HindIII, and inserted with the above hdcFv25 that is double digested by EcoRI, NotI, and thus the high efficient expression vector pTIH-hdcFv25 is constructed.

### Example 2

### Construction and Expression of RNase Expression Vector

As described below, the primers were optimally designed with reference to the full-length cDNA sequence of bullfrog RNase disclosed in the literature of Huang H. C. et al., Biochem 1998, 273 (11): 6395-7014, and with aid of the computer primer design software Premier 5.0 and Oligo:

P1: 5′ AAGCGGCCGCCTCAGAACTGGGCAACATT 3′ (SEQ ID NO: 9, wherein NotI restriction enzyme site is introduced, see underlined); and

P2: 5′ CCAAGCTTTGACAGCATGAAAACTAACTAAG 3′ (SEQ ID NO: 10, wherein Hind III restriction enzyme site is introduced, see underlined);

P3: 5′AAGGATCCCAGAACTGGGCAAC 3′ (SEQ ID NO: 11, wherein Bam HI restriction enzyme site is introduced, see underlined).

Total RNA was isolated from the liver of a female bullfrog. The cDNA was synthesized by using RT-PCR method, and bullfrog-RNase (RC-RNase being abbreviated as RNase) gene was amplified by using the above P1 and P2 primers, and then cloned into pUCm-T vector (purchased from Shanghai Bioasia), and thus pUCm-RNase recombinant plasmid was obtained. The plasmid was identified through NotI and Hind III double digestion, and was analyzed for the DNA sequence. The gene sequence is completely consistent with that reported by GeneBank, which indicates that the bullfrog RNase gene is successfully cloned.

Expression of RNase: With the pUCm-RNase recombinant plasmid as template, the gene fragments of RNase mature protein were encoded using PCR amplification, digested and inserted respectively into prokaryotic expression vectors that were similarly restriction enzyme digested, and thus formed the RNase fusion protein expression vector. The above two recombinant prokaryotic expression vectors, after being verified through restriction enzyme digestion and DNA sequencing test, were used to transform E. coli, induced by IPIG for expression, and the expressed product was analyzed by SDS-PAGE electrophoresis protein electrophoresis, and the target protein was determined by thin layer chromatography. The results showed that expression levels reached 12.5% and 34%, and the expressed product existed mainly in the form of inclusion body. The inclusion bodies, after being isolated and purified, were further purified by agarose affinity chromatography.

### Example 3

### Construction of Expression Vector pTIH-hdcFv25-RNase of Targeting Agents HdcFv25-RNase Fused Protein

With the cloning vector pUCm-RNase as template, and P4: 5′AAGCGGCCGCTCAGAACTGGGCAACATT 3′ (SEQ ID NO: 12) and P5: 5′AAGCGGCCGCTTAATGATGATGAT GATGATGACGCGGTTCCAGCGGATACGGCACCGGCGCACCAGGACATCGTC CTATTCCAGC 3′ as primers, PCR amplification was performed, and products were then applied with single enzyme digestion with NotI to form RNase. The above pTIH-hdcFv25 was similarly digested to obtain hdcFv25, connected by polymerase to form hHdcFv25-RNase fusion protein gene, cloned in expression vector pTIH, and thus became plasmid of pTIH-hdcFv25-RNase. After the sequence was verified, the product was transformed into competent bacteria E Coli and cultured, bacteria were lysised, and fusion proteins were isolated. Electrophoresis and Western Blot Analysis were performed, and the results indicate the expression of HdcFv25-RNase fusion protein.

### Example 4

### Construction, Expression and Purification of hdcFv25-PE38 and hscFv25-mTNF-α Fusion Proteins

1. Construction, Expression and Purification of hdcFv25-PE38

With the above expression vector pET15b-hdcFv25 as template, hdcFv25 was amplified, wherein hdcFv25 BACK: 5′ ATAGTTTAGCGGCCGCTTTGATCTCGA CCTGGTCCC3′ (SEQ ID NO: 14) and FOR: 5′ CGGAATTC ATGACCCAGACTCC ACTC 3′ (SEQ ID NO: 15) were used as primers. The PCR amplification products hdcFv25 was then double digested and DNA was recovered. With pCS18dPE38 as template, and PE BACK: 5′ GGAAGCTTTTAATGA TGATGATGATGATGCTTCAGGTCCTCGCGCGGCGG 3′ (SEQ ID NO: 16) and PE FOR: 5′ATAGTTTAGCGGCCGCTCAGGAG GGCGGCAGCCTGGCCGCG3′ (SEQ ID NO: 17) as primers, PE38 was amplified. The PCR products were identified in size by electrophoresis, and then double digested, and then DNA was recovered.

Polymerase was added to connect the above three digested fragments as pTIH-hdcFv25-PE38. This is a high efficient soluble expression vector, which was transformed into E. coli cells, and cultured in triple resistance LB medium that contains 200 mg/L ampicillin, 12.5 mg/L tetracycline, and 15 mg/L kanamycin, cultured with IPTG 0.1 mM at 30° C. for 4 hours to induce expression that occurs mainly in the supernatant. 2. Construction and Expression of HscFv25-mTNF-α

With pUC18-mTNF-α as template (mTNF-α for mutant TNF-α), and BACK 5′ ACGCGTCGACCGCAAACGTAAGCCTGTA 3′ and FOR: 5′ ACTCTGAGTCAGAAGGCAATGAT CCCAAAGT 3) as the upstream and downstream primers designed by using computer software Oligo, PCR amplification of mTNF-α was performed. The resultant product was connected with DNA ligase to form pGEX4T-1-scFv25-m-TNFα, transformed to competent E. Coli JM109. Monoclonal was picked and cultured at 37° C. overnight, and transferred in the Amp/LB medium and further cultured. IPTG induction was performed, bacteria were collected by centrifugation, lysozyme and sodium deoxycholate were added for lysis of bacteria, centrifuged, precipitated and washed 3 times. The inclusion bodies, after denaturation and renaturation, were purified by GST affinity chromatography. Western blot analysis with GST antibody (purchased from Pharmacia Company) was used to identify the target protein.

### Example 5

### In Vitro Potency Testing of Fusion Proteins

The activity of the fusion proteins was tested by MTT assay, and the results showed that the fusion proteins have excellent in vitro cytotoxicity. These fusion proteins were used in experimental treatment on nude mice with lever cancer by tail-vein injection once a day, each with 0.3 mL containing 20-30 μg, for 14 days. The results indicated that the inhibition rate was in the range of 75-79% as illustrated in Table 1 below.

TABLE 1
Results of Experimental treatments of Fusion Proteins Used to
Treat Nude Mice with Human Liver Cancer

| | Number of | Diameter of | |
|---|---|---|---|
| Fusion Protein | Nude Mice | Tumor | Remission Rate |
| scFV25-TNFα | 22 | 0.3 cm | 9 CR, 11 PR, 2 NR |
| hdcFv25-PE38 | 5 | 0.5 cm | 5 PR (77.0%) |
| hdcFv25-RNase | 5 | 0.5 cm | 5 PR (79.38%) |
| Doxorubicin (of same | 9 | 0.5 cm | 9 PR (42.0-70.0%) |
| dose) | | | |

CR: Complete remission;
PR: Partial remission;
NR: No remission.

### Example 6

### Encapsulation of Target Medicament HdcFv25-RNase Fusion Protein and RNase in Nano Liposomes

1. Preparation of Sterically Stabilized Liposomes: In chloroform were dissolved 100 mg of phospholipids and 25 mg of cholesterol (Chol) in a ratio of 4:1 (w/w), with cholesterol succinate. The chloroform was removed under vacuum by using a rotary film evaporator, and then an uniform dry film was formed on the sidewall of the flask.

2. Preparation of HdcFv25-RNase by Thin-Film Ultrasonic Method: Targeting agent was encapsulated by liposomes, and 2 mg/mL of HdcFv25—RC-RNase was prepared with a buffer of pH 7.0. Drug and lipid were quickly added into a flask, quickly mixed with vibratory rotation, and ultrasoned with an ultrasonic generator, filtered, granulated, and stored at 4° C. A Sephadex column (1.6 cm×30 cm) was loaded with 1 mL of liposomes, and run at flow rate of 0.5 mL/min. UV absorbance was measured with a 2 mL tube to distinguish between free HdcFv25-RNase and total HdcFv25-RNase, where the measured encapsulation ratio (E %) is:

$$E ( \% ) = ( 1 - \frac{C Free}{C Total} ) \times 100 \%$$

HdcFv25—RC-RNase was encapsulated with liposomes to form HdcFv25-RNase-Lp.

Rnase was similarly encapsulated to form RNase-Lp liposome. Double-targeting liposome hdcFv25-PE38-lp-hdcFv25 and control hdcFv25-PE38 and PE38-1p were also encapsulated.

### Example 7

### Crosslink of HdcFv25 on the Surface of Liposomes

To 1 mL of liposomes with hdcFv25-RNase encapsulated therein, was added 100 μL of S—NHS(N-hydroxysulfosuccinimide) then in the reaction liquid was added phospholipids and the reaction was kept at 4° C. overnight, so that hdcFv25 was cross-linked through the above reagents to the PEG molecules on the surface of the liposome to form hdcFv25-RNase-lp-hdcFv25.

### Example 8

### Test of Potency of the Drugs of This Invention to Cultured Hepatoma Cells

MTT essay is based on metabolic reduction of dimethylthiazol diphenyl tetrazolium bromide (MTT). The NAADP-related dehydrogenase existed in living cells is able to reduce yellow MTT to a blue-violet substance and thus can be used as an indicator of living cells. This enzyme, however, disappears in dead cells and thus is not able to reduce MIT, which remains in yellow. Thus, a micro plate reader was used to measure optical density at the wavelength of 550 nm to indicate the cell death rates, while IC50 refers to the dose where 50% of the tumor cells were inhibited.

Using MIT assay and the cultured hepatoma cells (SMMC-7721) as target tumor cells, anti-tumor activity tests were performed upon the double-targeting liposome hdcFv25-RNase-lp-hdcFv25 of the present invention, in comparison with the control RNase-lp-hdcFv25 liposome constructed in Example 5. The results are shown in Table 2.

TABLE 2
Comparison of MTT IC50 of hdcFv25-RNase-lp-hdcFv25 and
hdcFv25-RNase, RNase-lp

| | MTT IC50 | | MTT IC50 |
|---|---|---|---|
| | by weight | | by molar conc. |
| Dosage form | (μg/mL) | MW | (μM) |
| PBS | — | | |
| hdcFv25 | — | 26000 | |
| RNase | 53 | 14000 | 3.78 |
| hdcFv25-RNase | 53 | 40000 | 1.33 |
| RNase-lip-hdcFv25 | 38 | 40000 | 0.95 |
| hdcFv25-Rnase-lip-hdcFv25 | 30 | 66000 | 0.45 |

### Example 9

### Construction of the Double-Targeting Liposome of HdcFv25-PE38-lp-HdcFv25 and the Controls of HdcFv25-PE38 and PE38-lp and Anti-Tumor Activity Tests

The double-targeting liposome of HdcFv25-PE38-lp-HdcFv25 and the controls of HdcFv25-PE38 and PE38-lp were constructed as described in Examples 4 and 5, and a test was performed as described in Example 6. The test results are shown in Table 3.

TABLE 3
Comparison of MTT IC50 Reduction of HdcFv25-PE38-lp-HdcFv25,
and HdcFv25-PE38 and PE38-lp

| | MTT IC50 | |
|---|---|---|
| | by weight | |
| Dosage form | (μg/mL) | Reduction |
| PE38 | 92.07 | |
| hdcFv25-PE38 | 6.04 | |
| hdcFv25-PE38-lp-hdcFv25- | 0.59 | Reduced by 155.96 folds; |
| | | Reduced by 10.24 folds |

The IC50 dose was significantly reduced, indicating that the ability of killing tumor cells was markedly enhanced in the case of double-targeting liposome.

It can be assumed, not limited to any of particular theories, that the double-targeting liposome medicaments of the present invention enhance the targeted anti-tumor activities in a fashion as follows: HdcFv25 that is cross-linked onto the surface of the liposomes makes the liposomes and the HdcFv25-toxin (e.g., RNase) encapsulated therein to target to liver cancer cells; if the liposomes were damaged during delivery, the HdcFv25-toxin released from the liposomes still have targeting effect, the second targeting effect, so that the killing effect on tumor cells is enhanced.

The documents mentioned above are all incorporated herein by reference in their entirety as if each was incorporated individually. Further, it should be understood, after referring to the above disclosure of this invention, the ordinary skilled technicians in the art should be able to make various changes or modifications to the invention. These equivalent forms also fall into the scope of the attached claims of this application

What is claimed is:

1. A liposome medicament having targeting function, comprising a liposome and a medicament, wherein the medicament that is encapsulated inside of the liposome contains an effector, and a second ligand is combined on a surface of the liposome, and wherein the second ligand can specifically bind to target tissues or target cells of a subject to be treated or handled.

2. The liposome medicament of claim 1, wherein the medicament further contains an first ligand combined with the effector, and the first ligand can specifically bind to target tissues or target cells of a subject to be treated or handled.

3. The liposome medicament of claim 2, wherein the first ligand and the second ligand are the same.

4. The liposome medicament of claim 2, wherein the first ligand and the second ligand are different.

5. The liposome medicament of any of claims 2, wherein the first ligand and/or the second ligand, alone or both, are immunoglobulin, preferably a monoclonal antibody, and more preferably a specific antibody for tumor antigen.

6. The liposome medicament of claim 5, wherein the first ligand and the second ligand are independently a monoclonal antibody, Fab or F(ab′)₂ fragment of a monoclonal antibody, genetically engineered single-chain antibody scFv, or a humanized monoclonal antibody.

7. The liposome medicament of claim 6, wherein the antibody is disulfide bond stabilized.

8. The liposome medicament of claim 6, wherein the first ligand and/or the second ligand are independently selected from the group consisting of a single-chain antibody scFv25 having an amino acid sequence shown in SEQ ID NO: 1, a humanized single-chain antibody hscFv25 having an amino acid sequence shown in SEQ ID NO: 3, a humanized and disulfide-stabilized single-chain antibody HdcFv25 having an amino acid sequence shown in SEQ ID NO: 5, and a variant having an amino acid sequence that is different from SEQ ID NO: 1, 3 or 5 due to substitution, insertion, deletion and/or addition of 1-20, preferably 1-15, more preferably 1-10, even more preferably 1-8, particularly 1-5, for example 1, 2, 3 or 4 of amino acid residues.

9. The liposome medicament of claim 8, wherein the first ligand and/or the second ligand, either or both, are humanized and disulfide-stabilized anti-hepatoma single-chain antibody HdcFv25.

10. The liposome medicament of claim 1, wherein the effector is selected from the group consisting of toxin, medicament, enzyme, cytokine, radioisotope, chemotherapeutic agent, and tumor inhibiting gene.

11. The liposome medicament of claim 10, wherein the effector is a biotoxin, preferably being selected from the group consisting of diphtheria toxin of bacterial origin, pseudomonas exotoxin, ricin of plant origin, abrin, ribonuclease of other sources, phospholipase C, and complement, and more preferably being RNase or PE38 or mTNF.

12. The liposome medicament of claim 11, wherein the target medicament is a fusion protein of HdcFv25 and RNase or PE38 formed whether or not through fusion with linking peptide.

13. The liposome medicament of claim 1, wherein the second ligand is connected directly to the liposome, or is connected to the liposome through a chemical group, such as PEG, incorporated on the surface of the liposome.

14. The liposome medicament of claim 1, wherein the liposome comprises phospholipid (SPC) and cholesterol (Choi), with a ratio by weight of preferably 10:1 to 1:1, more preferably 8:1 to 2:1, even more preferably 6:1 to 3:1, and particularly 5:1 to 4:1.

15. The liposome medicament of claim 14, wherein optionally the liposome further incorporates therein a cholesterol derivative.

16. The liposome medicament of claim 15, wherein the liposome incorporates therein a cholesterol polyethylene glycol ester, in a ratio by molar number of phospholipids of 1-15%, preferably 3-10%, more preferably 5-8%, particularly 6%.

17. The liposome medicament of claim 1, wherein the liposome is a nanoliposome.

18. A method for preparing the liposome medicament of claim 1, comprising the steps of:
(1) Preparing a sterically stabilized liposome;
(2) Encapsulating inside of the liposome a targeting medicament containing an effector combined with a first ligand; and
(3) Cross-linking a second ligand on a surface of the liposome.

19. The method of claim 18, wherein the step (1) further comprises: Dissolving in chloroform a phospholipid and cholesterol in a desirable ratio by weight, and optionally a cholesterol polyethylene glycol ester, then removing the chloroform preferably under a reduced pressure to form a uniformed dry film of the liposomes.

20. The method of claim 18, wherein the step (2) further comprises: Constructing, expressing and purifying hdcFv25-RNase, hdcFv25-PE38, and hdcFv25-mTNF.

21. A method of treatment of diseases, comprising administering the liposome medicament of claim 1 or a medicament composition thereof to a patient in need of treatment.


SEQUENCE LISTING (19 sequences)

SEQ ID NO: 1 - protein, 253 aa, Artificial sequence
  Amino acid sequence of single-chain antibody scFv25
MQVQLLESGTELVKPGASVKLSCKASGYTFTSYWMHWVKQRPGQGLEWIGEIAPSNGRIYYNEKFKNKATLTVDKSSNTAYMELTSLTSEDSAVYSCARLVGPFSYWGQGTLVTVSSELGGGGSGGGGSGGGGSGSRCELVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTLGGGTKLELKRVD

SEQ ID NO: 2 - DNA, 759 bp, Artificial sequence
  Nucleotide sequence of encoding single-chain antibody scFv25
atgcaggtgcagctgttggagtctgggactgaactggtgaagcctggggcttcagtgaaactgtcctgcaaagcttctggctacaccttcaccagttattggatgcactgggtgaagcagaggcctggacaaggccttgagtggattggagagattaatcctagcaacggtcgtatttactacaatgaaaagttcaagaacaaggccacactgactgtagacaaatcctccaacacagcctatatggaactcaccagcctgacatctgaggactctgcggtctattcttgtgcaagattggtgggacccttctcttactggggccaaggcaccctggtcaccgtctcctcagagctcggtggaggcggttcaggtggaggtggctctggcggtggcggatctggatccagatgtgagctcgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttgggaggggggaccaaggtcgagatcaaacgtgtcgac

SEQ ID NO: 3 - protein, 244 aa, Artificial sequence
  Amino acid sequence of humanized single-chain antibody hscFv25
EFEVQLVESGGGLVQPGGSLKLSCAASGYTFTSYWMHWVRQGPGKGLEWIGEINPSNGRIYYNEKFKNKATLTVDKSKNTAYLQMNSLRAEDTVVYSCARLVGPFSYWGQGTLVTVSELGGGGSGGGGSGGGGSGSMTQTPLSLPVSLGDQASISCRSSQSLVHSNGNTYLHWYLQKPGQSPKLLIYKVSNRFSGVPDRFSGSGSGTDFTLEISRVEAEDLGVYFCSQSTHVPYTLGGTKVEIK

SEQ ID NO: 4 - DNA, 732 bp, Artificial sequence
  Nucleotide sequence of encoding humanized disulfide-stabilized single-chain antibody hdcFv25
gaattcgaggtacagctggttgaatctggtggtggtctggttcagccgggtggttctctgaaactgtcttgcgctgcttctggttacactttcacttcttactggatgcactgggttcgtcagggcccgggtaaaggtctggaatggatcggtgaaattaacccgtctaacggtcgtatctactacaacgaaaagttcaagaacaaagctactctgactgttgacaaatctaagaacactgcttacctgcagatgaactctctgcgtgctgaggacactgtggtctattcttgtgcaagattggtgggacccttctcttactggggctgtggcaccctggtcaccgtctcctaggagctcggtggaggcggttcaggtggaggtggctctggcggtggcggatctggatccatgacccagactccactctccctgcctgtcagtcttggagatcaagcctccatctcctgcagatctagtcagagccttgtacacagtaatggaaacacctatttacattggtacctgcagaagccaggccagtgtaagctcctgatctacaaagtttccaaccgattttctggggtcccagacaggttcagtggcagtggatcagggacagatttcacactcgagatcagcagagtggaggctgaggatctgggagtttatttctgctctcaaagtacacatgttccgtacacgttgggagggaccaaggtcgagatcaaa

SEQ ID NO: 5 - protein, 244 aa, Artificial sequence
  Amino acid sequence of humanized disulfide-stabilized single-chain antibody hdcFv25
EFEVQLVESGGGLVQPGGSLKLSCAASGYTFTSYWMHWVRQGPGKGLEWIGEINPSNGRIYYNEKFKNKATLTVDKSKNTAYLQMNSLRAEDTVVYSCARLVGPFSYWGCGTLVTVSELGGGGSGGGGSGGGGSGSMTQTPLSLPVSLGDQASISCRSSQSLVHSNGNTYLHWYLQKPGQCPKLLIYKVSNRFSGVPDRFSGSGSGTDFTLEISRVEAEDLGVYFCSQSTHVPYTLGGTKVEIK

SEQ ID NO: 6 - DNA, 40 bp, Artificial sequence
  Primer
ccgctcgacctggagacggtgaccaggatgcccagcccca

SEQ ID NO: 7 - DNA, 36 bp, Artificial sequence
  Primer
atagtttagcggccgctttgatctcgacctggtccc

SEQ ID NO: 8 - DNA, 26 bp, Artificial sequence
  Primer
cggaattcatgacccagactccactc

SEQ ID NO: 9 - DNA, 29 bp, Artificial sequence
  Primer
aagcggccgcctcagaactgggcaacatt

SEQ ID NO: 10 - DNA, 31 bp, Artificial sequence
  Primer
ccaagctttgacagcatgaaaactaactaag

SEQ ID NO: 11 - DNA, 22 bp, Artificial sequence
  Primer
aaggatcccagaactgggcaac

SEQ ID NO: 12 - DNA, 28 bp, Artificial sequence
  Primer
aagcggccgctcagaactgggcaacatt

SEQ ID NO: 13 - DNA, 85 bp, Artificial sequence
  Primer
aagcggccgcttaatgatgatgatgatgatgacgcggttccagcggatacggcaccggcgcaccaggacatcgtcctattccagc

SEQ ID NO: 14 - DNA, 36 bp, Artificial sequence; identical to SEQ ID NO: 7
  Primer

SEQ ID NO: 15 - DNA, 26 bp, Artificial sequence
  Primer
cggaattcatgacccagactccactc

SEQ ID NO: 16 - DNA, 50 bp, Artificial sequence
  Primer
ggaagcttttaatgatgatgatgatgatgcttcaggtcctcgcgcggcgg

SEQ ID NO: 17 - DNA, 41 bp, Artificial sequence
  Primer
atagtttagcggccgctcaggagggcggcagcctggccgcg

SEQ ID NO: 18 - DNA, 28 bp, Artificial sequence
  Primer
acgcgtcgaccgcaaacgtaagcctgta

SEQ ID NO: 19 - DNA, 31 bp, Artificial sequence
  Primer
actctgagtcagaaggcaatgatcccaaagt